(19) United States
(12) Patent Application Publication
(10) Pub. No.: US 2004/0131663 A1
(43) Pub. Date: Jul. 8, 2004

(54) Biodegradable wound dresssing

(76) Inventors: Alexander John Walacavage, Lansing, MI (US); John F. Holland, Haslett, MI (US)

Correspondence Address: John F. Holland, 1725 Maple Ridge, Unit 5, Haslett, MI, 48840, US

(21) Appl. No.: 10/675,722

(22) Filed: Sep. 30, 2003

Related U.S. Application Data
(60) Provisional application No. 60/414,255, filed on Sep. 30, 2002.

(51) Int. Cl. A61L 15/00; A61K 31/717

(52) U.S. Cl. 424/445; 514/60

(57) ABSTRACT

A hydrophilic pliable wound dressing consisting of bio-polymers creates a new approach to wound dressing by providing a self-assembling hydrophilic gel type moisture permeable barrier applicable to a wide range of wound sizes and depths. Biodegradability and water solubility enable rapid and safe disposal, obviating a significant problem that occurs with conventional type dressings. The initial sheet form of the dressing provides an ideal carrier for delivery of nutrients, debriders, antibiotics, analgesics and physiological regulators.

### BACKGROUND OF THE INVENTION

Wound healing presents a challenge to medical and surgical personnel. This is particularly true with large wounds having poorly defined edges; for example, burns, decubitus ulcers, venous stasis ulcers, arterial ulcers and serious abrasions. Wounds are often treated by covering them with products such as alginates, composites, contract layers, foams, hydrocolloids, hydrogels, impregnated gauzes, specialty absorptive, and transparent films. The theory behind the use of these products is that covering the wound decreases the risk of infection, keeps the wound from drying out, and decreases scarring.

Other products have been introduced which theoretically increase the rate of wound healing. Examples include topical cleansers, sealants, protectants, moisturizers, and enzymatic debriding agents. More recently, biological and biosynthetic dressings impregnated with collagen or related hydrolysates have been introduced. The theory behind these agents is that the collagen contained within the dressings mimics the natural collagen produced by the body and, thereby, promotes healing. Without treatment secreting skin wounds may lead to anemia, infections, shock and even death through the loss of body proteins, electrolytes, fluids and heat.

However, all of these approaches have shortcomings related to absorption characteristics and capacities, wound cleaning efficiency, discomfort in application and removal, bacterial susceptibility, expense and, in many cases, disposal problems. The proposed biodegradable starch foam dressing addresses and eliminates many of these shortcomings providing a self assembling gel barrier, which absorbs large quantities of exudates, maintains appropriate levels of hydration and allows body movement without restraint.

### SUMMARY OF THE INVENTION

A hydrophilic pliable wound dressing consisting of biopolymers provides a self-assembling hydrophilic gel serving as a moisture permeable barrier applicable to a wide range of wound sizes and depths. Biodegradability and water solubility enable rapid and safe disposal, obviating a significant problem that occurs with conventional type dressings. The initial sheet form of the dressing provides an ideal carrier for delivery of nutrients, enzymatic debriders antibiotics, analgesics and physiological regulators.

### BRIEF DESCRIPTION OF THE DRAWINGS

The drawings are simple and self-explanatory.

FIG. 1 shows the nature of the rolls produced and the sheets later cut from a roll.

FIG. 2 illustrates a band-aid type embodiment of the dressing.

FIG. 3 illustrates a tubular dispenser that can be used to apply a preformed gel to open wounds.

### DESCRIPTION OF THE INVENTION

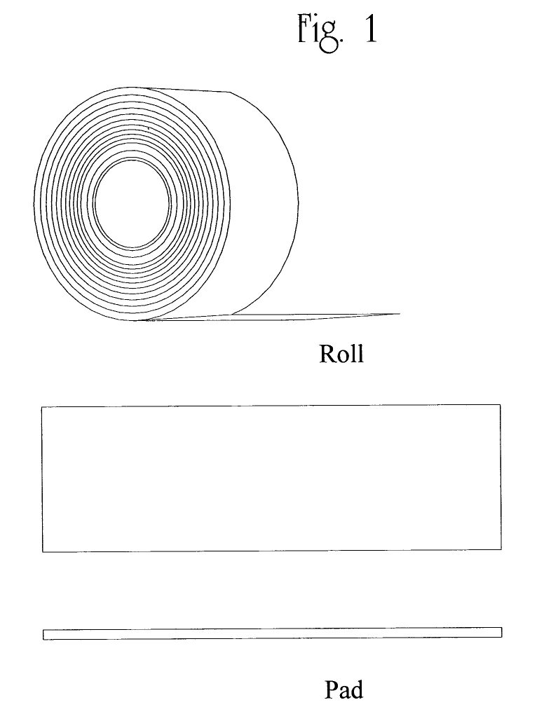

Natural carbohydrate polymers are extruded in a foam form in the shape of thin sheets. As shown in FIG. 1, these sheets may range in thickness from 1/32 inch to over an inch and in width ½ inch to 32 inches. As shown in the Figure, these sheets may be cut into various sizes and shapes to provide a wide range of dressings for a variety of wound sizes. These sizes can vary from one to a few inches on the sides of square, or rectangular shapes, to full body or appendage coverings of 1 to a few feet on each side.

Subsequent to cutting and sizing, the dressings are packaged antiseptically and made sterile. In application, the wound to be treated is thoroughly cleansed with saline, or a preferred topical agent, and any excess exudates removed and the wound debrided if necessary. An appropriate sized dressing is removed from its sterile package and placed over the wound. Larger sized dressings can be reduced in size to effectively match the area of the wound exposure. Upon contact with the moisture on the wound tissue the dressing turns into a gel creating a protective barrier that provides a moist environment for healing, absorbs drainage, exhibits chemo tactic action and provides topical nutrients. Upon removal, the dressing can be easily and rapidly dissolved by flushing with water and disposed of in a conventional drain.

In one embodiment of the invention, the biopolymer is composed of cornstarch polymerized with the assistance of an agent, such a poly vinyl alcohol (PVA). Other types of starch, such as potato, wheat, rice, etc. may be used as well as other polymerizing agents, such as poly ethylene glycol (PEG), poly propylene glycol (PPG), or one of several natural gums. In a preferred embodiment, the starch used is high amylose cornstarch extruded in flat sheet forms in a process using PVA as the polymerizing agent.

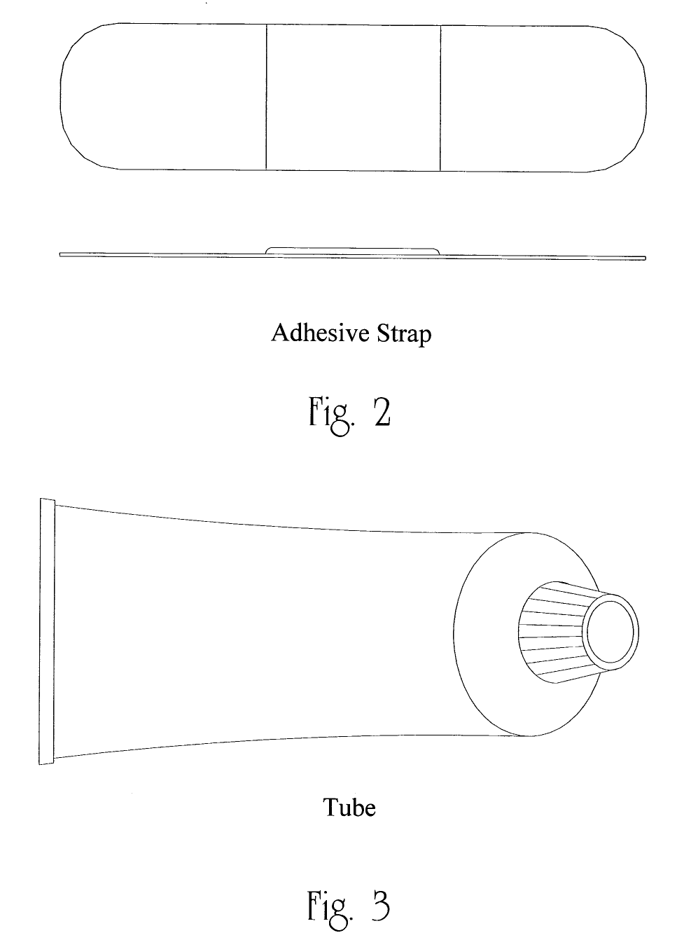

In another embodiment as shown in FIG. 2, the dressings are small and backed by an adhesive tape creating a novel band-aid type bandage that will provide the advantages of the biopolymer dressing to these applications. In a preferred embodiment, packaging is accomplished with commercial packaging wrappers and sterilization of the packaged dressing is accomplished by gamma radiation at levels sufficient for effective sterilization.

In normal situations adequate moisture is present in the wound to convert the polymer foam to the functioning gel. In situations where the natural moisture of a wound is very small, moisturizing the dressing with sterile saline or other agent prior to application may be required for convenience in fitting the dressing onto the wound site. In another embodiment of the invention, the starch foam dressing can be moistened to the point of a viscous fluid and applied to wound sites by dipping from an open container or extrusion from a flexible tube, as shown in FIG. 3. In another embodiment of the invention, the moisturizing agent is a pure water solution of nano-crystalline silver accomplishing both the role of hydrating the resulting gel and acting as a bactericide.

Wounds amenable for treatment with these dressings include acute abrasions, lacerations, burns, stage 2-4 pressure ulcers, diabetic ulcers, venous stasis ulcers, arterial ulcers, donor sites of skin grafts, post surgical incisions, appropriate dental applications and external wounds due to trauma.

### SUMMARY OF THE ADVANTAGES OF THE INVENTION

The advantageous features of this invention include effective linear wicking and the minimizing of pain and discomfort upon application and removal of the dressing. The gels created are non-toxic locally and systemically. The gel controls odors, decreases purulent exudates and are not absorbed systematically. The starch foams used are amenable to serve as vehicle for transport and delivery of amino acids, ascorbic acid and other nutrients; enzymatic debriders such as bromolin, and pupain; buffering compounds for pH regulation; and antibiotics, analgesics, bactericides and other compounds for treatment. Where appropriate, scented aromatics can also be incorporated into the foam. A major advantage lies in the ease of site removal and dressing disposal by simple washing and flushing procedures.

We claim:

1. A biodegradable hydrophilic starch polymer dressing for wound treatment.

2. A dressing in claim 1 wherein the starch polymer is dominantly comprised of the amylose form of the molecule.

3. A dressing in claim 1 in which the hydrophilic nature is optimally controlled.

4. A dressing in claim 1 which serves a vehicle of delivery of therapeutic agents, appropriate chemicals and biomolecules

5. A starch polymer that can be administered to wound sites as a preformed gel.
